(19) United States
(12) Patent Application Publication
(10) Pub. No.: US 2009/0132014 A1
(43) Pub. Date: May 21, 2009

(54) SELF FORMING TEMPERATURE TREATMENT PACK

(75) Inventors: Mark Kaminsky, Bloomfield Hills, MI (US); Robert John Vondell, Bloomfield Hills, MI (US)

Correspondence Address: GIFFORD, KRASS, SPRINKLE,ANDERSON & CITKOWSKI, P.C, PO BOX 7021, TROY, MI, 48007-7021, US

(73) Assignee: Flex-Ice, LLC, Troy, MI (US)

(21) Appl. No.: 12/271,232

(22) Filed: Nov. 14, 2008

Related U.S. Application Data
(60) Provisional application No. 60/988,115, filed on Nov. 15, 2007.

(51) Int. Cl. A61F 7/03; B29D 23/20

(52) U.S. Cl. 607/96; 156/198

(57) ABSTRACT

A temperature pack adapted to enclose an appendage, and method of forming such a temperature pack is provided. The temperature pack is a tubular pouch with an inner wall spaced apart from an outer wall. An opening extending through the tubular pouch, and the inner wall is twisted relative to the outer wall. The temperature pack is formed by rolling a tube onto itself such that the distal ends of the tube are aligned to each other. One distal end is then twisted with respect to the other so as to form a temperature pack having a mobious strip and only one seal. The temperature pack may be filled with a gelatinous solution capable of retaining heat or cold. Alternatively the temperature pack may be filled with particulate material separated from a liquid solvent wherein when the two are mixed an exothermic or endothermic reaction occurs.

### CROSS-REFERENCE TO RELATED APPLICATIONS

This application claims priority of U.S. Provisional Patent Application Ser. No. 60/988,115 filed Nov. 15, 2007, entitled “Self Forming Temperature Treatment Pack” which is incorporated herein by reference.

### FIELD OF THE INVENTION

The present invention relates to temperature packs. Specifically, the present invention relates to temperature packs adapted to enclose an appendage.

### BACKGROUND OF THE INVENTION

Temperature packs have many medical applications, among them is to reduce swelling and pain to a body part by subjecting the swollen body part to either hot or cold temperature. However, temperature packs are commonly rectangular in shape, or have a shape which does not allow the cold pack to remain fixed to a specific location without the aid of some other device such as medical tape.

The conventional rectangular shape of the temperature pack is thereby limiting because the shape will only apply direct temperature to the part of the appendage that the temperature pack is in contact with. Thus a portion of the appendage is without temperature treatment. For example, a broken finger would require at least two temperature packs so that the entire finger would be able to receive an equal treatment of temperature. Accordingly, it is desirable to have a temperature pack that can subject the entire appendage to temperature treatment at the same time without having the use of a fastening device, or an additional temperature pack.

### SUMMARY OF THE INVENTION

A temperature pack adapted to enclose an appendage, and a method of forming such a temperature pack is provided. The temperature pack is a tubular pouch with an inner wall spaced apart from an outer wall. The inner wall defines an opening extending through the tubular pouch and is twisted relative to the outer wall. The temperature pack is formed by rolling a tube onto itself such that the distal ends of the tube are aligned to each other. The distal ends are twisted with respect to each other, and the temperature pack is filled with an gelatinous solution. The distal ends are then sealed together.

The temperature pack my further include a frangible pouch disposed in the tubular pouch. The frangible pouch contains a liquid solvent. The tubular pouch is also is filled with particulate material that produces an exothermic or endothermic reaction with the contents of the frangible pouch. Accordingly, the frangible pouch is configured to rupture under a predetermined load so that the liquid solvent mixes with the particulate material so as to produce either an endothermic or exothermic reaction.

### BRIEF DESCRIPTION OF THE DRAWINGS

A better understanding of the present invention will be had upon reference to the following detailed description when read in conjunction with the accompanying drawing, wherein like reference characters refer to like parts throughout the several views, and in which:

FIG. 1 of the temperature pack with a mobius interior wall;

FIG. 1a is a view looking into the opening of FIG. 1;

FIG. 2 is an illustration of the temperature pack being applied onto a finger, the temperature pack rolls onto the finger and the mobius strip helps the temperature pack remain on the finger;

FIG. 3 is an illustration of the temperature pack having a frangible pouch, the frangible pouch separates liquid solvent from a particulate material, when the frangible pouch is broken the particulate material and liquid solvent mix and produce either an exothermic or endothermic reaction;

FIG. 4 is an illustration of temperature pack wherein the particulate material and liquid solvent are each enclosed in a frangible pouch;

FIGS. 5a-5d show the steps for forming a temperature pack with a mobius strip; and

FIG. 6 is an illustration of a temperature pack with a weakening point.

### DETAILED DESCRIPTION OF THE INVENTION

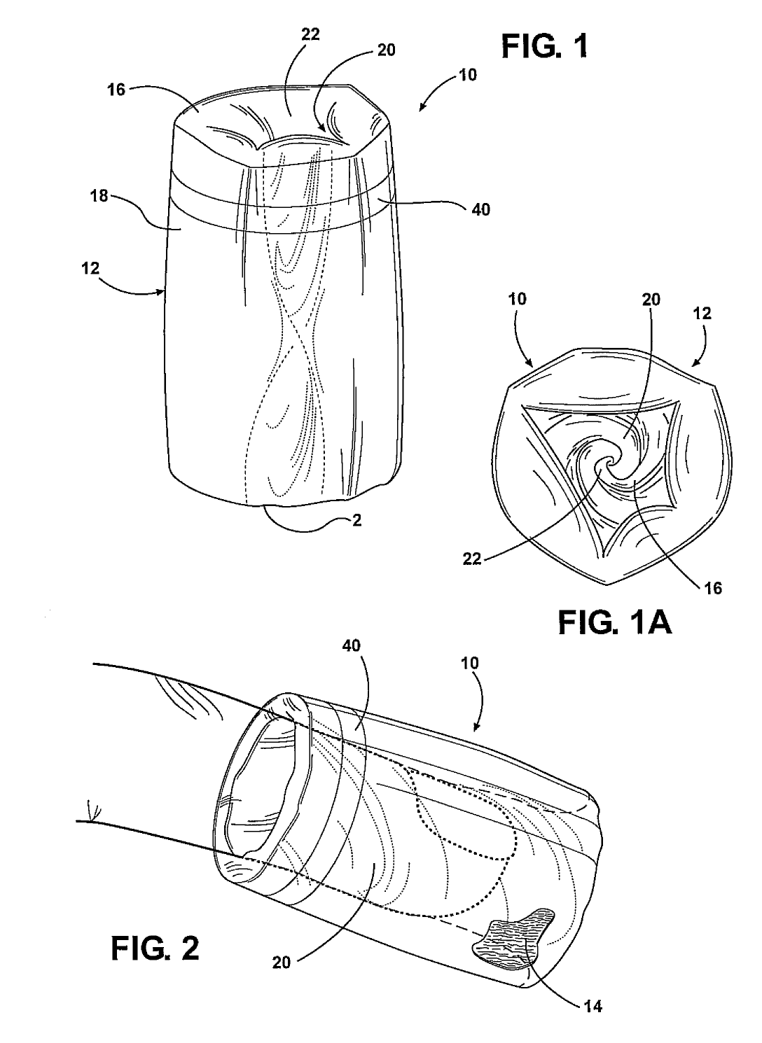

With reference to FIGS. 1 and 2, a tubular shaped self forming temperature pack 10 for use with a body appendage is provided. In a first preferred embodiment, the temperature pack 10 is a tubular pouch 12 containing a gelatinous solution 14 and is defined by an inner wall 16 that is twisted with respect to the outer wall 18 so as to form a mobius strip 20 in the inner wall 16. The temperature pack 10 has a first opening 22 spaced apart a second opening 24 so as to allow the temperature pack 10 to be rolled onto and enclose an appendage. Thus, it should be appreciated that the mobius inner wall 16 is continuously contorting as the temperature pack 10 is rolled onto an appendage.

The temperature pack 10 is formed from a tube 26. The tube 26 may be made of an elastomer having non-permeable qualities that can withstand a change in temperature without rupture. The tube 26 must also have elastic properties so as to better adapt to varying sizes of an appendage. A suitable elastomer may be synthetic or natural latex.

The body of the tube 26 has a first distal free end 28 and a second distal free end 30. The temperature pack 10 is formed by pulling one of the distal free ends 28, 30 over the body of the tube 26 and towards the other distal free end. Thus the tube 26 is placed in an overlapping relationship with itself so as to form a pocket 32. The pocket 32 can then be filled with the gelatinous solution 14. Alternatively, the pocket 32 may contain a particulate material 34 segregated from a liquid solvent 36 such that when the particulate material 34 comes into contact with the liquid solvent 36 an endothermic or exothermic reaction occurs.

The body of the tube 26 may be uniform in diameter. Alternatively, it is anticipated that the body of the tube 26 may be bow shaped so as to provide for a better pocket 32 for receiving material. The tube 26 further includes a mid-point 38 disposed between the first and second distal free ends 28, 30. The first and second distal free ends 28, 30 have generally the same diameter, while the diameter of the mid-point 38 is larger than that of the first and second distal free ends 28, 30. Thus, the body of the tube 26 widens as it extends from the first and second distal free ends 28, 30 to the mid-point 38 giving the tube 26 a bow shape.

The gelatinous solution 14 is capable of retaining heat at a relatively high temperature, or maintaining a relatively cold temperature. Such technology is known and is incorporated by reference herein. For example, U.S. Pat. No. 4,462,224 to Dunshee et al. discloses a cold pack having a gelatinous solution 14 being 70% water, 25% propylene glycol, and 5% hydroxypropyl methylcellulose. Other gelatinous solutions 14 are also known and generally contain a mixture of methylcellulose, alcohol, salt, or a water absorbent filler such as polyacrylamide. The gelatinous solution 14 may also contain a bactericide to help prevent mold. The gelatinous solution 14 is poured into the pocket 32, and the distal ends 28, 30 are twisted oppositely with respect to each other. The distal ends 28, 30 are then sealed together using a heat, adhesive, or the like. Thus a self forming temperature pack 10 with a mobius interior wall and only one seal 40 is formed. The self forming temperature pack 10 may be heated or cooled to a desired temperature and rolled onto an appendage. Furthermore, the temperature pack 10 may be reusable.

The temperature pack 10 is easily applied onto an appendage because the inner wall 16 of the temperature pack 10 may be rolled onto the appendage. Further, the mobius interior wall helps retain the temperature pack 10 onto an appendage because of the twisting and gripping action of the mobius interior wall on the appendage.

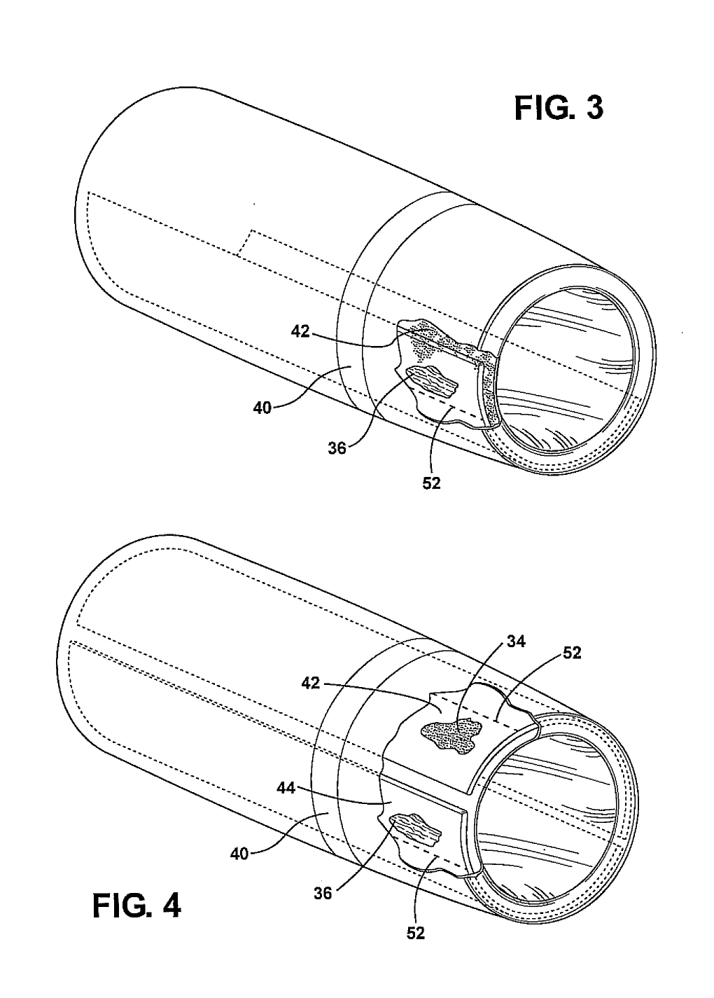

With reference now to FIGS. 3 and 4 a second preferred embodiment is provided. The temperature pack 10 contains a particulate material 34 segregated from a liquid solvent 36. Specifically, a frangible pouch 42 is disposed within the tubular pouch 12. The frangible pouch 42 is made of a material such as polypropylene and contains either the liquid solvent 36 or the particulate material 34.

The frangible pouch 42 may include a line of weakening 52 configured to break upon a predetermined load. The frangible pouch 42 serves to segregate the liquid solvent 36 from the particulate material 34. The frangible pouch 42 is configured to rupture upon experiencing a predetermined load. Any polypropylene known to those skilled in the art may be suitable for use in forming the frangible pouch 42, and the line of weakening 52 without deviating from the scope and spirit of the present invention.

It is also anticipated that the frangible pouch 42 may be made of two different polypropylene sealed together, wherein one polypropylene has a greater material strength than the other. Thus a line of weakening 52 is formed along the seal so as to rupture the frangible pouch 42 upon a predetermined load.

Alternatively, the temperature pack 10 may include a second frangible pouch 44 such that the liquid solvent 36 and particulate material 34 are each contained in a separate pouch. Thus it should be inherent that the strength of the material of the tube 26 should be greater than that of the material of the frangible pouch 42.

In operation, the temperature pack 10 is initially at room temperature. The user merely exerts a predetermined load onto the temperature so as to rupture the frangible pouch 42 therein. The pouch will then begin to get hot or cold as the particulate material 34 and solvent come into contact with each other.

Instant hot/cold technology is currently known and used and is incorporated by reference herein. For instance, U.S. Pat. No. 4,081,256 to Donnelly discloses a particulate material 34 producing an endothermic reaction when mixed with water. The particulate material 34 disclosed in Donnelly consists of, in parts by weight, about 65 to about 130 parts urea, about 35 to about 80 parts hydrated sodium acetate (Na C₂H₃O₂ 3H₂O), about 18 to about 40 parts potassium chloride, potassium nitrate, or mixtures thereof, about 18 to about 30 parts ammonium chloride, and about 6 to about 10 parts quar gum. It is anticipated that other heat-absorbing particulate material 34 such as ammonium nitrate crystals may be used. It is common knowledge that ammonium nitrate crystals produce an endothermic chemical reaction when mixed with a liquid solvent 36.

Alternatively, the particulate material 34 may be a heat-producing particulate material 34 such as dry salt, anhydrous magnesium sulfate, calcium chloride, or the like. For instance, U.S. Pat. No. 4,067,313 to Donnelly discloses a heat-producing particulate material 34 consisting of, in parts by weight, about 40 to about 90 parts anhydrous calcium chloride, about 12 to about 25 parts anhydrous sodium acetate, and about 5 to parts 15 calcium oxide. The heat-producing particulate material 34 produces an exothermic reaction when it comes into contact with a liquid solvent 36 such as water. It is commonly understood by those in the art that the liquid solvent 36 may contain other ingredients such as coloring, or an inorganic salt, such as sodium chloride.

It is anticipated that the temperature pack 10 may include a liner 46. The liner 46 may be formed from any material that helps promote the therapeutic values of temperature treatment. For instance, the temperature pack 10 may be lined with cloth or felt in order to provide moist heat application to the treated appendage, or to prevent a cold treatment from causing frost bite on the treated appendage.

Furthermore, the temperature pack 10 may come in different sizes to accommodate different body appendages. Naturally, a temperature pack 10 for treating a finger is going to be smaller than a temperature pack 10 for treating a foot. The temperature pack 10 may also come with different features such as a neck support for use on the shoulder. In such an embodiment, an inflatable pouch may be attached to the temperature pack 10 so as to support a neck.

The temperature pack 10 may also include a weakening point 48, as shown in FIG. 6. For instance an aperture 50 may be fitted within the filled tubular temperature pack 10 in order to accommodate the bending of a joint connecting two appendages such as a knee or an elbow. The weakening point 48 may be formed by simply cutting two holes in the tube 26. The tube 26 may then be pulled over itself and the holes are aligned to each other. The edges of the holes are sealed together so as to form an aperture 50 in the temperature pack 10.

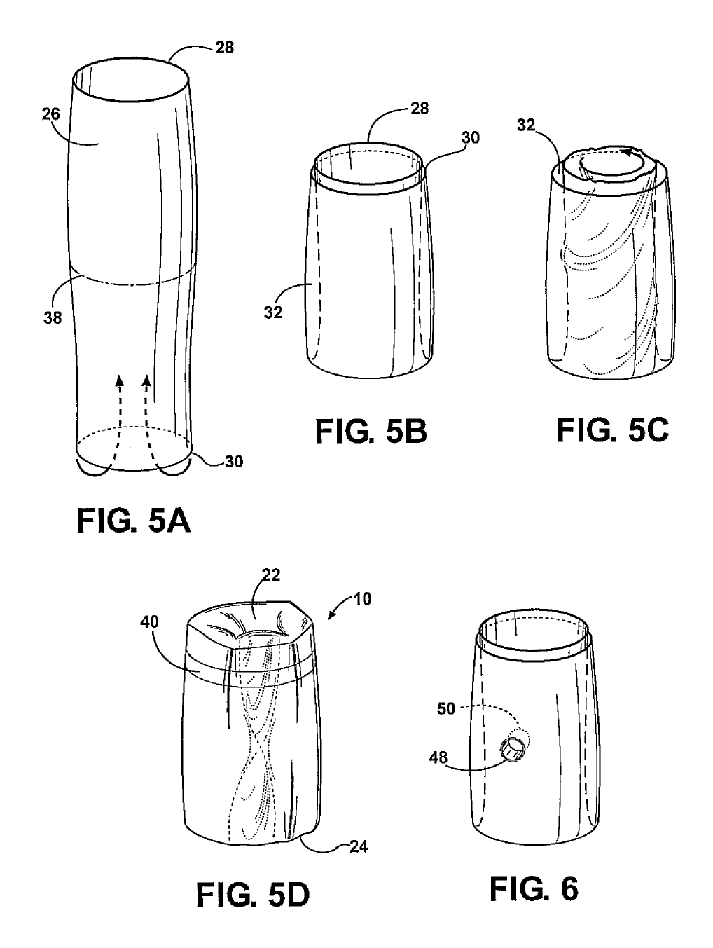

With reference now to FIGS. 5a-5d, a method of forming a temperature pack 10 is provided. The method includes providing a tube 26. The tube 26 has a first distal free end 28 opposite a second distal free end 30. In the next step of the method, the tube 26 is rolled over itself such that the first distal free end 28 is aligned with the second distal free end 30. The body of the tube 26 is now placed in an overlapping relationship with itself so as to form a tubular pouch 12 with an opening extending therethrough. The first distal free end 28 is twisted with respect to the second distal free end 30 so as to form a mobius strip 20. The tubular pouch 12 may be filled either before or after the mobius strip 20 is formed. In the last step of the method, the first and second distal free ends 28, 30 are sealed together. Thus a temperature pack 10 having only one seal 40 and a mobius strip 20 is formed.

A method of forming a temperature pack 10 capable rolling onto and engaging an appendage as well as producing an exothermic or endothermic reaction is also provided. The method includes providing a tube 26. The tube 26 has a first distal free end 28 opposite a second distal free end 30. In the next step of the method, the tube 26 is rolled over itself such that the first distal free end 28 is aligned with the second distal free end 30. The body of the tube 26 is now placed in an overlapping relationship with itself so as to form a tubular pouch 12 with an opening extending therethrough A frangible pouch 42 configured to rupture upon experiencing the predetermined load is also provided. The frangible pouch 42 is filled with either a liquid solvent 36 or a particulate material 34. The tubular pouch 12 is filled with the other of a liquid solvent 36 or particulate material 34. The frangible pouch 42 is inserted into the filled tubular pouch 12 and the distal free ends 28, 30 are sealed together. Thus a temperature pack 10 having only one seal 40 and capable of producing either an endothermic or exothermic reaction is provided, wherein the temperature pack 10 is configured to roll onto an engage an appendage.

Obviously many modifications and variations of the temperature pack 10 disclosed are possible in light of the above description and may be practiced otherwise than as specifically described while within the scope of the above described temperature pack 10.

We claim:

1. A temperature pack comprising:
a tubular pouch having an inner wall spaced apart from an outer wall, the inner wall defines an opening extending through the tubular pouch so as to allow the temperature pack to be rolled onto and engage an appendage;
an gelatinous solution contained within the tubular pouch; and
wherein the inner wall and outer wall are twisted with respect to each other so as to form a mobius strip, and wherein the free end of the inner wall is sealed to the free end of the outer wall so as to form a seal enclosing the gelatinous solution within the tubular pouch.

2. A temperature pack as set forth in claim 1 wherein the temperature pack includes a weakening point so as to accommodate the flex of a body appendage.

3. A temperature pack as set forth in claim 2 wherein the weakening point is an aperture.

4. A temperature pack as set forth in claim 1 wherein the inner wall includes a first opening and the outer wall includes a second opening, and wherein the first opening is scaled to the second opening after the inner wall has been twisted relative to the outer wall.

5. A temperature pack as set forth in claim 1 wherein the tubular pouch includes a first distal free end opposite a second distal free end, and a mid-point disposed therebetween, and wherein the first and second distal free ends have generally the same diameter, and the diameter of the mid-point is larger than that of the first and second distal free ends.

6. A temperature pack as set forth in claim 1 further including a liner.

7. A temperature pack as set forth in claim 1 wherein the inner wall defines the mobius strip.

8. A temperature pack as set forth in claim 1 wherein the tubular pouch is formed from latex.

9. A temperature pack as set forth in claim 1 wherein the free end of the inner wall and the free end of the outer wall are sealed together by one selected from the group consisting of adhesives, thermal seal, or ultrasonic seal.

10. A temperature pack comprising:
a tubular pouch having an inner wall spaced apart from an outer wall, the inner wall defines an opening extending through the tubular pouch so as to allow the temperature pack to be rolled onto and engage an appendage;
a particulate material disposed in the tubular pouch;
a frangible pouch also disposed within the tubular pouch, the frangible pouch containing a liquid solvent, and wherein the frangible pouch is ruptured upon a predetermined load so as release the liquid solvent, and wherein the liquid solvent upon contact with the heat-absorbing material begins to cool the pouch to a predetermined temperature.

11. A temperature pack as set forth in claim 10 wherein the tubular pouch is made from latex.

12. A temperature pack as set forth in claim 10 wherein the frangible pouch is made from polypropylene.

13. A temperature pack as set forth in claim 10 wherein the particulate material is heat-absorbing.

14. A temperature pack as set forth in claim 10 wherein the particulate material is heat-producing.

15. A temperature pack as set forth in claim 10 further including a second frangible pouch disposed within the tubular pouch, and wherein the second frangible pouch contains the particulate material, and wherein the second frangible pouch ruptures upon experiencing the predetermined load so as to cause the particulate material to come into contact with the liquid solvent.

16. A temperature as set forth in claim 10 having only one seal.

17. A method for manufacturing a temperature pack having a mobius inner wall, the method of manufacturing the temperature pack comprising the steps of:
providing a tube having a first distal free end opposite a second distal free end;
rolling the tube over itself such that the first distal free end is aligned with the second distal free end so as to form a pouch with an opening extending therethrough;
filling the pouch with an gelatinous solution;
twisting the first distal free end with respect to the second distal free end; and
sealing the first distal free end to the second distal free end so as to form a temperature pack with a mobius inner wall.

18. A method for manufacturing a tubular temperature pack, wherein the tubular temperature pack is configured to roll onto an appendage and produces chemical reaction when experiencing a predetermined load, the method of manufacturing the tubular temperature pack comprising the steps of:
providing a tube having a first distal free end opposite a second distal free end;
rolling the tube over itself such that the first distal free end is aligned with the second distal free end so as to form a pouch with an opening extending therethrough;
filling the pouch with an particulate material;
providing a frangible pouch configured to rupture upon experiencing the predetermined load, wherein the frangible pouch is filled with a liquid solvent;
inserting the frangible pouch in the pouch; and
sealing the first distal free end to the second distal free end so as to form a tubular temperature pack.

19. A method as set forth in claim 18 wherein the particulate material produces an endothermic reaction when mixed with the liquid solvent.

20. A method as set forth in claim 18 wherein the particulate material produces an exothermic reaction when mixed with the liquid solvent.
